(19) United States
(12) Patent Application Publication
(10) Pub. No.: US 2026/0257068 A1
(43) Pub. Date: Sep. 3, 2026

(54) IMPROVED PROGRAMMING OF NEURAL STIMULATION THERAPY

(71) Applicant: Saluda Medical Pty Ltd, Macquarie Park, New South Wales (AU)

(72) Inventors: Matthew Marlon Williams, Macquarie Park (AU); Dean Michael Karantonis, Macquarie Park (AU); Daniel John Parker, Macquarie Park (AU)

(21) Appl. No.: 18/832,036

(22) PCT Filed: Jan. 30, 2023
(86) PCT No.: PCT/AU2023/050049

§ 371 Date: Jul. 22, 2024

(30) Foreign Application Priority Data
Jan. 28, 2022 (AU) 2022900147

(51) Int. Cl. A61N 1/36; A61N 1/02; A61N 1/05; A61N 1/372; G16H 20/30

(52) U.S. Cl. CPC A61N 1/36139; A61N 1/025; A61N 1/36125; A61N 1/36185; A61N 1/37235; G16H 20/30; A61N 1/0551; A61N 1/36062

(57) ABSTRACT

Disclosed is a neural stimulation system comprising a neural stimulation device for controllably delivering a neural stimulus, and a processor. The processor is configured to: instruct a control unit of the neurostimulation device to control a stimulus source to sequentially deliver a plurality of neural stimuli via respective stimulus electrode configurations of a plurality of stimulus electrode configurations according to respective values of a stimulus intensity parameter, wherein the value of the stimulus intensity parameter at each stimulus electrode configuration is different from a preceding value of the stimulus intensity parameter at that stimulus electrode configuration; receive a captured signal window corresponding to each delivered neural stimulus; measure an intensity of an evoked neural response in each captured signal window, thereby forming a plurality of (stimulus intensity parameter, response intensity) pairs for each stimulus electrode configuration; and estimate one or more key parameters of an activation plot at each stimulus electrode configuration, using the plurality of (stimulus intensity parameter, response intensity) pairs for each stimulus electrode configuration.

The present application claims priority from Australian Provisional Patent Application No 2022900147 filed on 28 Jan. 2022, the contents of which are incorporated herein by reference in their entirety.

### TECHNICAL FIELD

The present invention relates to neural stimulation therapy and in particular to programming a neural stimulation therapy system to suit the needs of a particular patient.

### BACKGROUND OF THE INVENTION

There are a range of situations in which it is desirable to apply neural stimuli in order to alter neural function, a process known as neuromodulation. For example, neuromodulation is used to treat a variety of disorders including chronic neuropathic pain, Parkinson's disease, and migraine. A neuromodulation system applies an electrical pulse (stimulus) to neural tissue (fibres, or neurons) in order to generate a therapeutic effect. In general, the electrical stimulus generated by a neuromodulation system evokes a neural response known as an action potential in a neural fibre which then has either an inhibitory or excitatory effect. Inhibitory effects can be used to modulate an undesired process such as the transmission of pain, or excitatory effects may be used to cause a desired effect such as the contraction of a muscle.

When used to relieve neuropathic pain originating in the trunk and limbs, the electrical pulse is applied to the dorsal column (DC) of the spinal cord, a procedure referred to as spinal cord stimulation (SCS). Such a system typically comprises an implanted electrical pulse generator, and a power source such as a battery that may be transcutaneously rechargeable by wireless means, such as inductive transfer. An electrode array is connected to the pulse generator, and is implanted adjacent the target neural fibre(s) in the spinal cord, typically in the dorsal epidural space above the dorsal column. An electrical pulse of sufficient intensity applied to the target neural fibres by a stimulus electrode causes the depolarisation of neurons in the fibres, which in turn generates an action potential in the fibres. Action potentials propagate along the fibres in orthodromic (towards the head, or rostral) and antidromic (towards the cauda, or caudal) directions. The fibres being stimulated in this way inhibit the transmission of pain from a region of the body innervated by the target neural fibres (the dermatome) to the brain. To sustain the pain relief effects, stimuli are applied repeatedly, for example at a frequency in the range of 30 Hz-100 Hz.

For effective and comfortable neuromodulation, it is necessary to maintain stimulus intensity above a recruitment threshold. Stimuli below the recruitment threshold will fail to recruit sufficient neurons to generate action potentials with a therapeutic effect. In almost all neuromodulation applications, response from a single class of fibre is desired, but the stimulus waveforms employed can evoke action potentials in other classes of fibres which cause unwanted side effects. In pain relief, is therefore necessary to apply stimuli with intensity below a discomfort threshold, above which uncomfortable or painful percepts arise due to over-recruitment of Aβ fibres. When recruitment is too large, Aβ fibres produce uncomfortable sensations. Stimulation at high intensity may even recruit Aδ fibres, which are sensory nerve fibres associated with acute pain, cold and pressure sensation. It is therefore desirable to maintain stimulus intensity within a therapeutic range between the recruitment threshold and the discomfort threshold.

The task of maintaining appropriate neural recruitment is made more difficult by electrode migration (change in position over time) and/or postural changes of the implant recipient (patient), either of which can significantly alter the neural recruitment arising from a given stimulus, and therefore the therapeutic range. There is room in the epidural space for the electrode array to move, and such array movement from migration or posture change alters the electrode-to-fibre distance and thus the recruitment efficacy of a given stimulus. Moreover, the spinal cord itself can move within the cerebrospinal fluid (CSF) with respect to the dura. During postural changes, the amount of CSF and/or the distance between the spinal cord and the electrode can change significantly. This effect is so large that postural changes alone can cause a previously comfortable and effective stimulus regime to become either ineffectual or painful.

Attempts have been made to address such problems by way of feedback or closed-loop control, such as using the methods set forth in International Patent Publication No. WO2012/155188 by the present applicant. Feedback control seeks to compensate for relative nerve/electrode movement by controlling the intensity of the delivered stimuli so as to maintain a substantially constant neural recruitment. The intensity of a neural response evoked by a stimulus may be used as a feedback variable representative of the amount of neural recruitment. A signal representative of the neural response may be generated by a measurement electrode in electrical communication with the recruited neural fibres, and processed to obtain the feedback variable. Based on the response intensity, the intensity of the applied stimulus may be adjusted to maintain the response intensity within a therapeutic range.

It is therefore desirable to accurately measure the intensity and other characteristics of a neural response evoked by the stimulus. The action potentials generated by the depolarisation of a large number of fibres by a stimulus sum to form a measurable signal known as an evoked compound action potential (ECAP). Accordingly, an ECAP is the sum of responses from a large number of single fibre action potentials. The ECAP generated from the depolarisation of a group of similar fibres may be measured at a measurement electrode as a positive peak potential, then a negative peak, followed by a second positive peak. This morphology is caused by the region of activation passing the measurement electrode as the action potentials propagate along the individual fibres.

Approaches proposed for obtaining a neural response measurement are described by the present applicant in International Patent Publication No. WO 2012/155183, the content of which is incorporated herein by reference.

However, neural response measurement can be a difficult task as an observed CAP signal component in the measured response will typically have a maximum amplitude in the range of microvolts. In contrast, a stimulus applied to evoke the CAP is typically several volts, and manifests in the measured response as crosstalk of that magnitude. Moreover, stimulus generally results in electrode artefact, which manifests in the measured response as a decaying output of the order of several millivolts after the end of the stimulus. As the CAP signal can be contemporaneous with the stimulus crosstalk and/or the stimulus artefact, CAP measurements present a difficult challenge of measurement amplifier design. For example, to resolve a 10 μV CAP with 1 μV resolution in the presence of stimulus crosstalk of 5 V requires an amplifier with a dynamic range of 134 dB, which is impractical in implantable devices. In practice, many non-ideal aspects of a circuit lead to artefact, and as these aspects mostly result a time-decaying artefact waveform of positive or negative polarity, their identification and elimination can be laborious.

Closed-loop neural stimulation therapy is governed by a number of parameters to which values must be assigned to implement the therapy. The effectiveness of the therapy depends in large measure on the suitability of the assigned parameter values to the patient undergoing the therapy. As patients vary significantly in their physiological characteristics, a “one-size-fits-all” approach to parameter value assignment is likely to result in ineffective therapy for a large proportion of patients. An important preliminary task, once a neuromodulation device has been implanted in a patient, is therefore to assign values to the therapy parameters that maximise the effectiveness of the therapy the device will deliver to that particular patient. This task is known as programming or fitting the device. Programming generally involves applying certain test stimuli via the device, recording responses, and based on the recorded responses, inferring or calculating the most effective parameter values for the patient. The resulting parameter values are then formed into a “program” that may be loaded to the device to govern subsequent therapy. Some of the recorded responses may be neural responses evoked by the test stimuli, which provide an objective source of information that may be analysed along with subjective responses elicited from the patient. In an effective programming system, the more responses that are analysed, the more effective the eventual assigned parameter values should be.

However, programming may be costly and time-consuming if unnecessarily prolonged. There is therefore an incentive to minimise the number of test stimuli to be applied and the amount of information to be recorded and analysed in order to produce the assigned values of the therapy parameters. Moreover, thresholds for discomfort vary widely between patients, between postures for a single patient, and between stimulus electrodes for a given patient in a given posture. It is difficult to know in advance where a given patient's discomfort threshold is in a given posture. The result is that a test stimulus of an intensity that is comfortable for one patient may provoke acute discomfort for another patient, or for the same patient in a different posture, or for the same patient in the same posture when applied at a different stimulus electrode. This complicates certain aspects of programming involving measurement of the intensity of patients' neural responses across the full comfortable range of stimulus intensity at a particular stimulus electrode.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In this specification, a statement that an element may be “at least one of” a list of options is to be understood to mean that the element may be any one of the listed options, or may be any combination of two or more of the listed options.

### SUMMARY OF THE INVENTION

Disclosed herein are methods and devices for rapidly estimating key parameters of a patient's activation plot at multiple stimulus locations without causing discomfort. Single stimulus pulses of varying intensities in the therapeutic range are applied in interleaved fashion in rapid succession at all of the stimulus locations to be measured. Respective ECAP amplitudes, or response intensities, are measured in response to each respective stimulus pulse. The cycle is iterated multiple times to obtain a set of (stimulus intensity, response intensity) pairs over the therapeutic range for each stimulus location. The key parameters may be estimated at a given stimulus location from the set of (stimulus intensity, response intensity) pairs.

According to a first aspect of the present technology, there is provided a neurostimulation system comprising: a neurostimulation device for controllably delivering a neural stimulus, and a processor. The neurostimulation device comprises: a plurality of implantable electrodes including one or more stimulus electrodes and one or more sense electrodes, wherein a stimulus electrode configuration comprises at least one stimulus electrode acting as an anode and at least one stimulus electrode acting as a cathode; a stimulus source configured to deliver neural stimuli via a stimulus electrode configuration to a neural pathway of a patient; measurement circuitry configured to capture signal windows sensed at a sense electrode of the one or more sense electrodes in response to respective neural stimuli; and a control unit configured to control the stimulus source to deliver each neural stimulus according to a stimulus intensity parameter. The processor is configured to: instruct the control unit to control the stimulus source to sequentially deliver a plurality of neural stimuli via respective stimulus electrode configurations of a plurality of stimulus electrode configurations according to respective values of the stimulus intensity parameter, wherein the value of the stimulus intensity parameter at each stimulus electrode configuration is different from a preceding value of the stimulus intensity parameter at that stimulus electrode configuration; receive a captured signal window corresponding to each delivered neural stimulus; measure an intensity of an evoked neural response in each captured signal window, thereby forming a plurality of (stimulus intensity parameter, response intensity) pairs for each stimulus electrode configuration; and estimate one or more key parameters of an activation plot at each stimulus electrode configuration, using the plurality of (stimulus intensity parameter, response intensity) pairs for each stimulus electrode configuration.

According to a second aspect of the present technology, there is provided an automated method of estimating one or more key parameters of neural responses evoked by neural stimuli delivered to a patient. The method comprises: sequentially delivering a plurality of neural stimuli via respective stimulus electrode configurations of a plurality of stimulus electrode configurations according to respective values of a stimulus intensity parameter, wherein the value of the stimulus intensity parameter at each stimulus electrode configuration is different from a preceding value of the stimulus intensity parameter at that stimulus electrode configuration; receiving a captured signal window corresponding to each delivered neural stimulus; measuring an intensity of an evoked neural response in each captured signal window, thereby forming a plurality of (stimulus intensity parameter, response intensity) pairs for each stimulus electrode configuration; and estimating one or more key parameters of an activation plot at each stimulus electrode configuration, using the plurality of (stimulus intensity parameter, response intensity) pairs for each stimulus electrode configuration.

According to a third aspect of the present technology, there is provided a neural stimulation system comprising: a neural stimulation device for controllably delivering a neural stimulus, and a processor. The neurostimulation device comprises: a plurality of implantable electrodes including one or more stimulus electrodes and one or more sense electrodes, wherein a stimulus electrode configuration comprises at least one stimulus electrode acting as an anode and at least one stimulus electrode acting as a cathode; a stimulus source configured to deliver neural stimuli via a stimulus electrode configuration to a neural pathway of a patient; measurement circuitry configured to capture signal windows sensed at a sense electrode of the one or more sense electrodes in response to respective neural stimuli; and a control unit configured to control the stimulus source to deliver each neural stimulus according to a stimulus intensity parameter. The processor is configured to: instruct the control unit to control the stimulus source to sequentially deliver a plurality of neural stimuli via respective stimulus electrode configurations of a plurality of stimulus electrode configurations according to respective values of the stimulus intensity parameter, wherein each value of the stimulus intensity parameter at each stimulus electrode configuration is different from a preceding value of the stimulus intensity parameter at that stimulus electrode configuration; receive a captured signal window corresponding to each delivered neural stimulus; determine whether an evoked neural response is present in each captured signal window; and estimate an ECAP threshold at each stimulus electrode configuration using the determinations of whether an evoked neural response is present in each captured signal window.

According to a fourth aspect of the present technology, there is provided an automated method of estimating a key parameter of neural responses evoked by neural stimuli delivered to a patient. The method comprises: sequentially delivering a plurality of neural stimuli via respective stimulus electrode configurations of a plurality of stimulus electrode configurations according to respective values of a stimulus intensity parameter, wherein each value of the stimulus intensity parameter at each stimulus electrode configuration is different from a preceding value of the stimulus intensity parameter at that stimulus electrode configuration; receiving a captured signal window corresponding to each delivered neural stimulus; determining whether an evoked neural response is present in each captured signal window; and estimating an ECAP threshold at each stimulus electrode configuration using the determinations of whether an evoked neural response is present in each captured signal window.

The phenomenon of psychophysical masking means that stimulus pulses of what would be uncomfortably high intensity if repeated at a single stimulus location do not cause discomfort if delivered in isolation at that stimulus location, when interleaved with other stimulus pulses at other intensities and/or when interleaved with other stimulus pulses at other stimulus locations. The discomfort of this process in such embodiments of the present invention may therefore be reduced as compared to other methods in which the key parameters are estimated at each stimulus location separately in time from those at other stimulus locations.

In some embodiments of the invention, the processor is configured to instruct the control unit to control the stimulus source to sequentially deliver a first plurality of neural stimuli via a first stimulus electrode configuration according to respective values of the stimulus intensity parameter; and instruct the control unit to control the stimulus source to sequentially deliver, interleaved with the first plurality of neural stimuli, a second plurality of neural stimuli via a second stimulus electrode configuration according to respective values of the stimulus intensity parameter.

In some embodiments, the stimuli may be delivered from the respective stimulus electrode configurations in an order which is the same when repeated. For example, the order may be configured to maximize a geometric distance between consecutively used stimulus electrode configurations.

In some embodiments, the stimuli may be delivered from the respective stimulus electrode configurations in an order which is permuted when repeated. For example Markov sampling may be used to permute the order. A transition probability matrix of the Markov sampling may be altered, depending on whether a preceding response intensity was zero.

In some embodiments, the value of the stimulus intensity parameter may be chosen between an ECAP threshold T and a discomfort threshold Max for the corresponding stimulus electrode configuration. The value of the stimulus intensity parameter may be sampled from a uniform distribution between the ECAP threshold T and the discomfort threshold Max for the corresponding stimulus electrode configuration. The value of the stimulus intensity parameter may be sampled from respective normal distributions each having parameters which, at a respective stimulus electrode configuration, depend dynamically on measurements from one or more preceding stimulus electrode configurations. A mean and a standard deviation of the respective normal distribution at each stimulus electrode configuration may be initially set to a default and, after stimulating at a preceding stimulus electrode configuration, the mean of the distribution at a neighbouring stimulus electrode configuration may be reduced if a non-zero response intensity was detected from the preceding stimulus electrode configuration or may be increased if a zero response intensity was detected on the preceding stimulus electrode configuration. After stimulating at the preceding stimulus electrode configuration, the standard deviation of the neighbouring stimulus electrode configuration distribution may be altered by an amount which is a function of the geometric distance between the stimulus electrode configurations. The discomfort threshold at each stimulus electrode configuration may be estimated from the ECAP threshold at that stimulus electrode configuration. The discomfort threshold at each stimulus electrode configuration may be estimated by applying a linear model to the ECAP threshold at that stimulus electrode configuration. The ECAP threshold at each stimulus electrode configuration may be estimated. For example by applying a noise departure detector to detect ECAPs in captured signal windows corresponding to multiple neural stimuli of different stimulus intensity parameters delivered via the stimulus electrode configuration.

In some embodiments, one or more key parameters at each stimulus electrode configuration may be estimated by fitting an activation plot model to the plurality of (stimulus intensity parameter, response intensity) pairs for that stimulus electrode configuration. The activation plot model may be a straight line, and one of the one or more key parameters may be a slope of the fitted straight line and/or an intercept of the fitted straight line. The activation plot model may be a logistic growth curve, and one of the one or more key parameters may be a slope of the fitted logistic growth curve at its midpoint.

In some embodiments, the processor may be part of the neural stimulation device. The system may further comprise an external computing device in communication with the neural stimulation device. The processor may be part of the external computing device.

In some embodiments, a histogram of stimulus intensity values at which evoked neural responses were determined to be present may be formed. The histogram values may be normalised. The ECAP threshold at each stimulus electrode configuration may be estimated using the normalised histogram for that stimulus electrode configuration. The ECAP threshold at each stimulus electrode configuration may be estimated by interpolating the normalised histogram values to find the intensity at which the ECAP detection rate is 50%.

References herein to estimation, determination, comparison and the like are to be understood as referring to an automated process carried out on data by a processor operating to execute a predefined procedure suitable to effect the described estimation, determination and/or comparison step(s). The technology disclosed herein may be implemented in hardware (e.g., using digital signal processors, application specific integrated circuits (ASICs) or field programmable gate arrays (FPGAs)), or in software (e.g., using instructions tangibly stored on non-transitory computer-readable media for causing a data processing system to perform the steps described herein), or in a combination of hardware and software. The disclosed technology can also be embodied as computer-readable code on a computer-readable medium. The computer-readable medium can include any data storage device that can store data which can thereafter be read by a computer system. Examples of the computer-readable medium include read-only memory (“ROM”), random-access memory (“RAM”), magnetic tape, optical data storage devices, flash storage devices, or any other suitable storage devices. The computer-readable medium can also be distributed over network-coupled computer systems so that the computer-readable code is stored and/or executed in a distributed fashion.

### BRIEF DESCRIPTION OF THE DRAWINGS

One or more implementations of the invention will now be described with reference to the accompanying drawings, in which:

FIG. 1 schematically illustrates an implanted spinal cord stimulator, according to one implementation of the present technology;

FIG. 2 is a block diagram of the stimulator of FIG. 1;

FIG. 3 is a schematic illustrating interaction of the implanted stimulator of FIG. 1 with a nerve;

FIG. 4a illustrates an idealised activation plot for one posture of a patient undergoing neural stimulation;

FIG. 4b illustrates the variation in the activation plots with changing posture of the patient;

FIG. 5 is a schematic illustrating elements and inputs of a closed-loop neural stimulation system, according to one implementation of the present technology;

FIG. 6 illustrates the typical form of an electrically evoked compound action potential (ECAP) of a healthy subject;

FIG. 7 is a block diagram of a neural stimulation therapy system including the implanted stimulator of FIG. 1 according to one implementation of the present technology;

FIG. 8 is a flow chart illustrating a method of estimating key parameters of the patient's response to stimulus at multiple SECs simultaneously;

FIG. 9 is a flow chart illustrating a method of using the noise departure detector to estimate the ECAP threshold at each SEC, for use in the method of FIG. 8;

FIG. 10 is a flow chart illustrating a method of using the noise departure detector to estimate the ECAP threshold at each SEC, for use in the method of FIG. 8; and

FIG. 11 is an illustration of a timing sequence of stimulus pulse delivery according to one implementation of the present technology.

### DETAILED DESCRIPTION OF THE PRESENT TECHNOLOGY

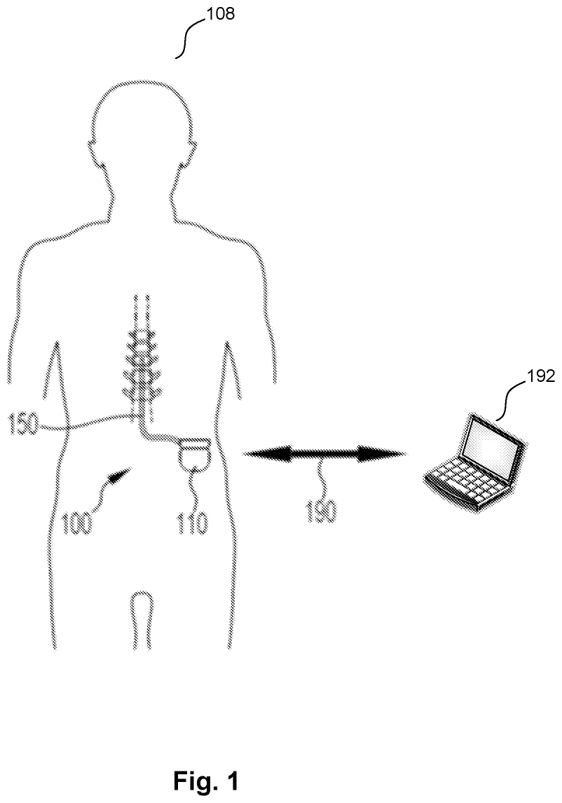

FIG. 1 schematically illustrates an implanted spinal cord stimulator 100 in a patient 108, according to one implementation of the present technology. Stimulator 100 comprises an electronics module 110 implanted at a suitable location. In one implementation, stimulator 100 is implanted in the patient's lower abdominal area or posterior superior gluteal region. In other implementations, the electronics module 110 is implanted in other locations, such as a flank or sub-clavicularly. Stimulator 100 further comprises an electrode array 150 implanted within the epidural space and connected to the module 110 by a suitable lead. The electrode array 150 may comprise one or more electrodes such as electrode pads on a paddle lead, circular (e.g., ring) electrodes surrounding the body of the lead, conformable electrodes, cuff electrodes, segmented electrodes, or any other type of electrodes capable of forming unipolar, bipolar or multipolar electrode configurations for stimulation and measurement. The electrodes may pierce or affix directly to the tissue itself.

Numerous aspects of the operation of implanted stimulator 100 may be programmable by an external computing device 192, which may be operable by a user such as a clinician or the patient 108. Moreover, implanted stimulator 100 serves a data gathering role, with gathered data being communicated to external device 192 via a transcutaneous communications channel 190. Communications channel 190 may be active on a substantially continuous basis, at periodic intervals, at non-periodic intervals, or upon request from the external device 192. External device 192 may thus provide a clinical interface configured to program the implanted stimulator 100 and recover data stored on the implanted stimulator 100. This configuration is achieved by program instructions collectively referred to as the Clinical Programming Application (CPA) and stored in an instruction memory of the clinical interface.

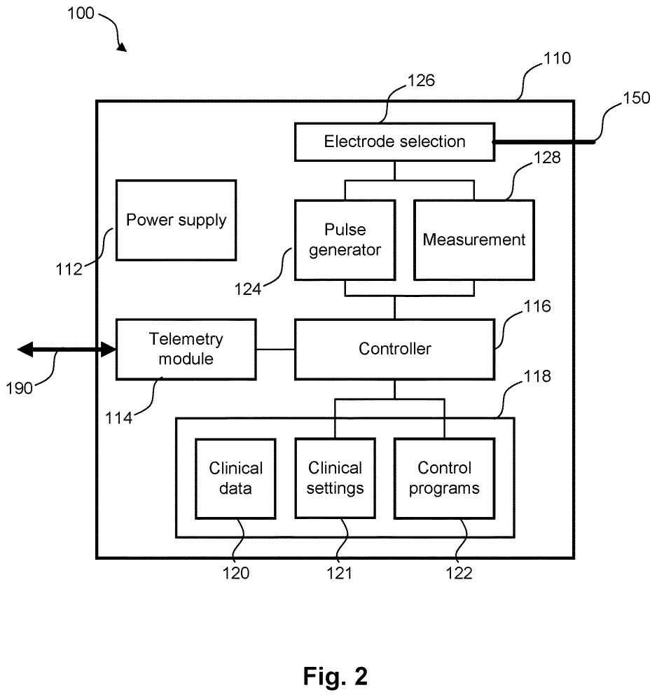

FIG. 2 is a block diagram of the stimulator 100. Electronics module 110 contains a battery 112 and a telemetry module 114. In implementations of the present technology, any suitable type of transcutaneous communications channel 190, such as infrared (IR), radiofrequency (RF), capacitive and/or inductive transfer, may be used by telemetry module 114 to transfer power and/or data to and from the electronics module 110 via communications channel 190. Module controller 116 has an associated memory 118 storing one or more of clinical data 120, clinical settings 121, control programs 122, and the like. Controller 116 controls a pulse generator 124 to generate stimuli, such as in the form of pulses, in accordance with the clinical settings 121 and control programs 122. Electrode selection module 126 switches the generated pulses to the selected electrode(s) of electrode array 150, for delivery of the pulses to the tissue surrounding the selected electrode(s). Measurement circuitry 128, which may comprise an amplifier and/or an analog-to-digital converter (ADC), is configured to process signals comprising neural responses sensed at measurement electrode(s) of the electrode array 150 as selected by electrode selection module 126.

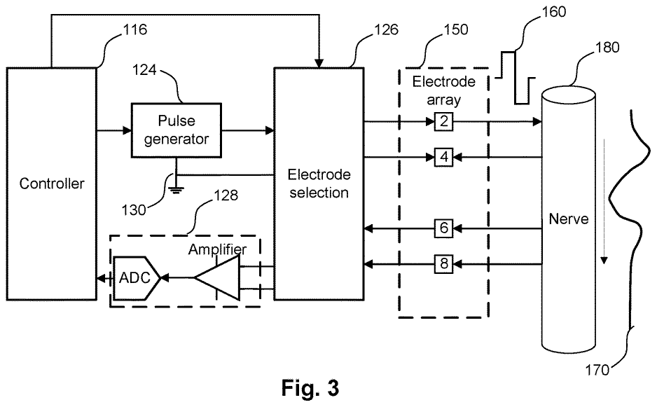

FIG. 3 is a schematic illustrating interaction of the implanted stimulator 100 with a nerve 180 in the patient 108. In the implementation illustrated in FIG. 3 the nerve 180 may be located in the spinal cord, however in alternative implementations the stimulator 100 may be positioned adjacent any desired neural tissue including a peripheral nerve, visceral nerve, parasympathetic nerve or a brain structure. Electrode selection module 126 selects a stimulus electrode 2 of electrode array 150 through which to deliver a pulse from the pulse generator 124 to surrounding tissue including nerve 180. A pulse may comprise one or more phases, e.g. a biphasic stimulus pulse 160 comprises two phases. Electrode selection module 126 also selects a return electrode 4 of the electrode array 150 for stimulus current return in each phase, to maintain a zero net charge transfer. An electrode may act as both a stimulus and a return electrode over a complete multiphasic stimulus pulse. The use of two electrodes in this manner for delivering and returning current in each stimulus phase is referred to as bipolar stimulation. Alternative embodiments may apply other forms of bipolar stimulation, or may use a greater number of stimulus and/or return electrodes. In all cases, the set of stimulus electrodes and return electrodes is referred to as the stimulus electrode configuration (SEC). Electrode selection module 126 is illustrated as connecting to a ground 130 of the pulse generator 124 to enable stimulus current return via the return electrode 4. However, other connections for charge recovery may be used in other implementations.

Delivery of an appropriate stimulus from electrodes 2 and 4 to the nerve 180 evokes a neural response 170 comprising an evoked compound action potential (ECAP) which will propagate along the nerve 180 as illustrated at a rate known as the conduction velocity. The ECAP may be evoked for therapeutic purposes, which in the case of a spinal cord stimulator for chronic pain may be to create paraesthesia at a desired location. To this end, the electrodes 2 and 4 are used to deliver stimuli periodically at any therapeutically suitable frequency, for example 30 Hz, although other frequencies may be used including frequencies as high as the kHz range. In alternative implementations, stimuli may be delivered in a non-periodic manner such as in bursts, or sporadically, as appropriate for the patient 108. To program the stimulator 100 to the patient 108, a clinician may cause the stimulator 100 to deliver stimuli of various configurations which seek to produce a sensation that is experienced by the user as paraesthesia. When a stimulus electrode configuration is found which evokes paraesthesia in a location and of a size which is congruent with the area of the patient's body affected by pain, the clinician nominates that configuration for ongoing use. The therapy parameters may be loaded into the memory 118 of the stimulator 100 as the clinical settings 121.

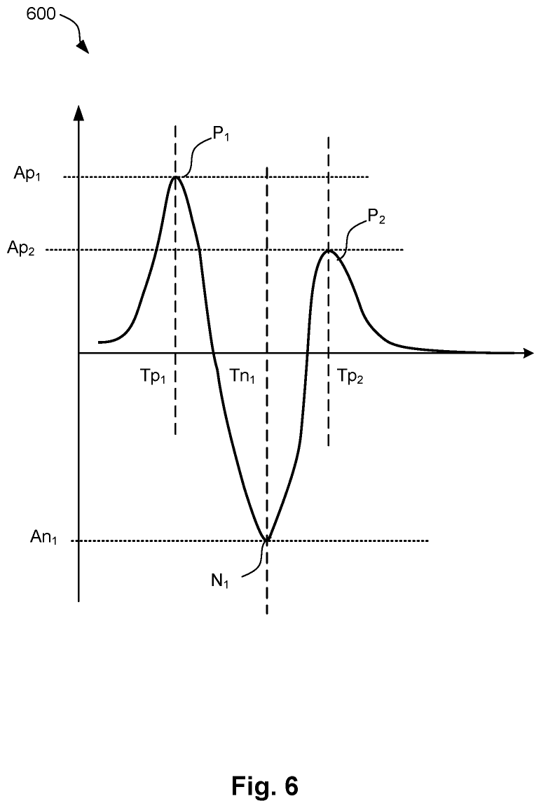

FIG. 6 illustrates the typical form of an ECAP 600 of a healthy subject, as recorded at a single measurement electrode referenced to the system ground 130. The shape and duration of the ECAP 600 shown in FIG. 6 is predictable because it is a result of the ion currents produced by the ensemble of fibres depolarising and generating action potentials (APs) in response to stimulation. The evoked action potentials (EAPs) generated synchronously among a large number of fibres sum to form the ECAP 600. The ECAP 600 generated from the synchronous depolarisation of a group of similar fibres comprises a positive peak P1, then a negative peak N1, followed by a second positive peak P2. This shape is caused by the region of activation passing the measurement electrode as the action potentials propagate along the individual fibres.

The ECAP may be recorded differentially using two measurement electrodes, as illustrated in FIG. 3. Depending on the polarity of recording, a differential ECAP may take an inverse form to that shown in FIG. 6, i.e. a form having two negative peaks N1 and N2, and one positive peak P1. Alternatively, depending on the distance between the two measurement electrodes, a differential ECAP may resemble the time derivative of the ECAP 600, or more generally the difference between the ECAP 600 and a time-delayed copy thereof.

The ECAP 600 may be characterised by any suitable characteristic(s) of which some are indicated in FIG. 6. The amplitude of the positive peak P1 is Ap₁ and occurs at time Tp₁. The amplitude of the positive peak P2 is Ap₂ and occurs at time Tp₂. The amplitude of the negative peak P1 is An₁ and occurs at time Tn₁. The peak-to-peak amplitude is Ap₁+An₁. A recorded ECAP will typically have a maximum peak-to-peak amplitude in the range of microvolts and a duration of 2 to 3 ms.

The stimulator 100 is further configured to detect the existence and measure the intensity of ECAPs 170 propagating along nerve 180, whether such ECAPs are evoked by the stimulus from electrodes 2 and 4, or otherwise evoked. To this end, any electrodes of the array 150 may be selected by the electrode selection module 126 to serve as recording electrode 6 and reference electrode 8, whereby the electrode selection module 126 selectively connects the chosen electrodes to the inputs of the measurement circuitry 128. Thus, signals sensed by the measurement electrodes 6 and 8 subsequent to the respective stimuli are passed to the measurement circuitry 128, which may comprise a differential amplifier and an analog-to-digital converter (ADC), as illustrated in FIG. 3. The recording electrode and the reference electrode are referred to as the measurement electrode configuration. The measurement circuitry 128 for example may operate in accordance with the teachings of the above-mentioned International Patent Application Publication No. WO2012/155183.

Signals sensed by the measurement electrodes 6, 8 and processed by measurement circuitry 128 are further processed by an ECAP detector implemented within controller 116, configured by control programs 122, to obtain information regarding the effect of the applied stimulus upon the nerve 180. In some implementations, the sensed signals are processed by the ECAP detector in a manner which measures and stores one or more characteristics from each evoked neural response or group of evoked neural responses contained in the sensed signal. In one such implementation, the characteristics comprise a peak-to-peak ECAP amplitude in microvolts (μV). For example, the sensed signals may be processed by the ECAP detector to determine the peak-to-peak ECAP amplitude in accordance with the teachings of International Patent Publication No. WO2015/074121, the contents of which are incorporated herein by reference. Alternative implementations of the ECAP detector may measure and store an alternative characteristic from the neural response, or may measure and store two or more characteristics from the neural response.

Stimulator 100 applies stimuli over a potentially long period such as days, weeks, or months and during this time may store characteristics of neural responses, stimulation settings, paraesthesia target level, and other operational parameters in memory 118. To effect suitable SCS therapy, stimulator 100 may deliver tens, hundreds or even thousands of stimuli per second, for many hours each day. Each neural response or group of responses generates one or more characteristics such as a measure of the intensity of the neural response. Stimulator 100 thus may produce such data at a rate of tens or hundreds of Hz, or even kHz, and over the course of hours or days this process results in large amounts of clinical data 120 which may be stored in the memory 118. Memory 118 is however necessarily of limited capacity and care is thus required to select compact data forms for storage into the memory 118, to ensure that the memory 118 is not exhausted before such time that the data is expected to be retrieved wirelessly by external device 192, which may occur only once or twice a day, or less.

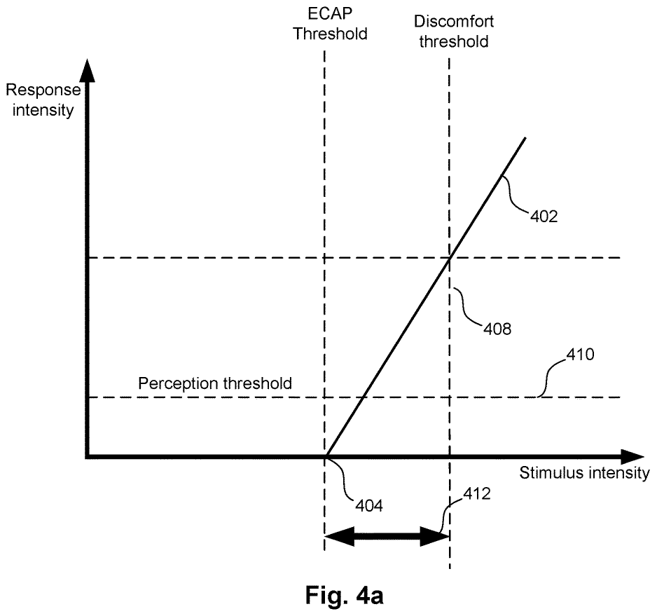

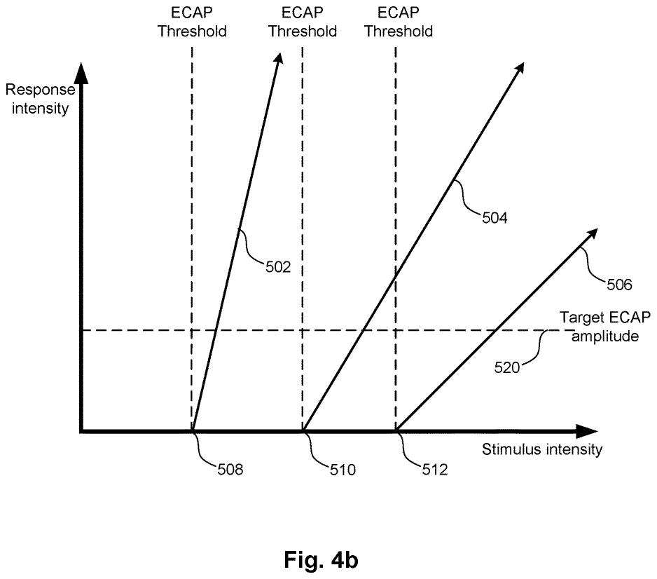

An activation plot, or growth curve, is an approximation to the relationship between stimulus intensity (e.g. an amplitude of the current pulse 160) and intensity of neural response 170 resulting from the stimulus (e.g. a peak-to-peak ECAP amplitude). FIG. 4a illustrates an idealised activation plot 402 for one posture of the patient 108. The activation plot 402 shows a linearly increasing ECAP amplitude for stimulus intensity values above a recruitment threshold also referred to as the ECAP threshold 404. The ECAP threshold exists because of the binary nature of fibre recruitment; if the field strength is too low, no fibres will be recruited. However, once the field strength exceeds a threshold, fibres begin to be recruited, and their individual evoked action potentials are independent of the strength of the field. The ECAP threshold 404 therefore reflects the field strength at which significant numbers of fibres begin to be recruited, and the increase in response intensity with stimulus intensity above the ECAP threshold reflects increasing numbers of fibres being recruited. Below the ECAP threshold 404, the ECAP amplitude may be taken to be zero. Above the ECAP threshold 404, the activation plot 402 has a positive, approximately constant slope indicating a linear relationship between stimulus intensity and the ECAP amplitude. Such a relationship may be modelled as:

$$y = \{ \begin{matrix}S ( s - T ) , & s \geq T \\ 0 , & s < T\end{matrix} \qquad ( 1 )$$

where s is the stimulus intensity, y is the ECAP amplitude, Tis the ECAP threshold and S is the slope of the activation plot (referred to herein as the patient sensitivity). The slope S and the ECAP threshold T are the key parameters of the activation plot 402.

FIG. 4a also illustrates a discomfort threshold 408, which is a stimulus intensity above which the patient 108 experiences uncomfortable or painful stimulation. FIG. 4a also illustrates a perception threshold 410. The perception threshold 410 corresponds to an ECAP amplitude that is perceivable by the patient. There are a number of factors which can influence the position of the perception threshold 410, including the posture of the patient. Perception threshold 410 may correspond to a stimulus intensity that is greater than the ECAP threshold 404, as illustrated in FIG. 4a, if patient 108 does not perceive low levels of neural activation. Conversely, the perception threshold 410 may correspond to a stimulus intensity that is less than the ECAP threshold 404, if the patient has a high perception sensitivity to lower levels of neural activation than can be detected in an ECAP, or if the signal to noise ratio of the ECAP is low.

For effective and comfortable operation of an implantable neuromodulation device such as the stimulator 100, it is desirable to maintain stimulus intensity within a therapeutic range. A stimulus intensity within a therapeutic range 412 is above the ECAP threshold 404 and below the discomfort threshold 408. In principle, it would be straightforward to measure these limits and ensure that stimulus intensity, which may be closely controlled, always falls within the therapeutic range 412. However, the activation plot, and therefore the therapeutic range 412, varies with the posture of the patient 108.

FIG. 4b illustrates the variation in the activation plots with changing posture of the patient. A change in posture of the patient may cause a change in impedance of the electrode-tissue interface or a change in the distance between electrodes and the neurons. While the activation plots for only three postures, 502, 504 and 506, are shown in FIG. 4b, the activation plot for any given posture can lie between or outside the activation plots shown, on a continuously varying basis depending on posture. Consequently, as the patient's posture changes, the ECAP threshold changes, as indicated by the ECAP thresholds 508, 510, and 512 for the respective activation plots 502, 504, and 506. Additionally, as the patient's posture changes, the slope of the activation plot also changes, as indicated by the varying slopes of activation plots 502, 504, and 506. In general, as the distance between the stimulus electrodes and the spinal cord increases, the ECAP threshold increases and the slope of the activation plot decreases. The activation plots 502, 504, and 506 therefore correspond to increasing distance between stimulus electrodes and spinal cord, and decreasing patient sensitivity.

To keep the applied stimulus intensity within the therapeutic range as patient posture varies, in some implementations an implantable neuromodulation device such as the stimulator 100 may adjust the applied stimulus intensity based on a feedback variable that is determined from one or more measured ECAP characteristics. In one implementation, the device may adjust the stimulus intensity to maintain the measured ECAP amplitude at a target response intensity. For example, the device may calculate an error between a target ECAP amplitude and a measured ECAP amplitude, and adjust the applied stimulus intensity to reduce the error as much as possible, such as by adding the scaled error to the current stimulus intensity. A neuromodulation device that operates by adjusting the applied stimulus intensity based on a measured ECAP characteristic is said to be operating in closed-loop mode and will also be referred to as a closed-loop neural stimulus (CLNS) device. By adjusting the applied stimulus intensity to maintain the measured ECAP amplitude at an appropriate target response intensity, such as an ECAP target 520 illustrated in FIG. 4b, a CLNS device will generally keep the stimulus intensity within the therapeutic range as patient posture varies.

A CLNS device comprises a stimulator that takes a stimulus intensity value and converts it into a neural stimulus comprising a sequence of electrical pulses according to a predefined stimulation pattern. The stimulation pattern is parametrised by multiple stimulus parameters including stimulus amplitude, pulse width, number of phases, order of phases, number of stimulus electrode poles (two for bipolar, three for tripolar etc.), and stimulus rate or frequency. At least one of the stimulus parameters, for example the stimulus amplitude, is controlled by the feedback loop.

In an example CLNS system, a user (e.g. the patient or a clinician) sets a target response intensity, and the CLNS device performs proportional-integral-differential (PID) control. In some implementations, the differential contribution is disregarded and the CLNS device uses a first order integrating feedback loop. The stimulator produces stimulus in accordance with a stimulus intensity parameter, which evokes a neural response in the patient. The intensity of an evoked neural response (e.g. an ECAP) is detected, and its amplitude measured by the CLNS device and compared to the target response intensity.

The measured neural response intensity, and its deviation from the target response intensity, is used by the feedback loop to determine possible adjustments to the stimulus intensity parameter to maintain the neural response at the target intensity. If the target intensity is properly chosen, the patient receives consistently comfortable and therapeutic stimulation through posture changes and other perturbations to the stimulus/response behaviour.

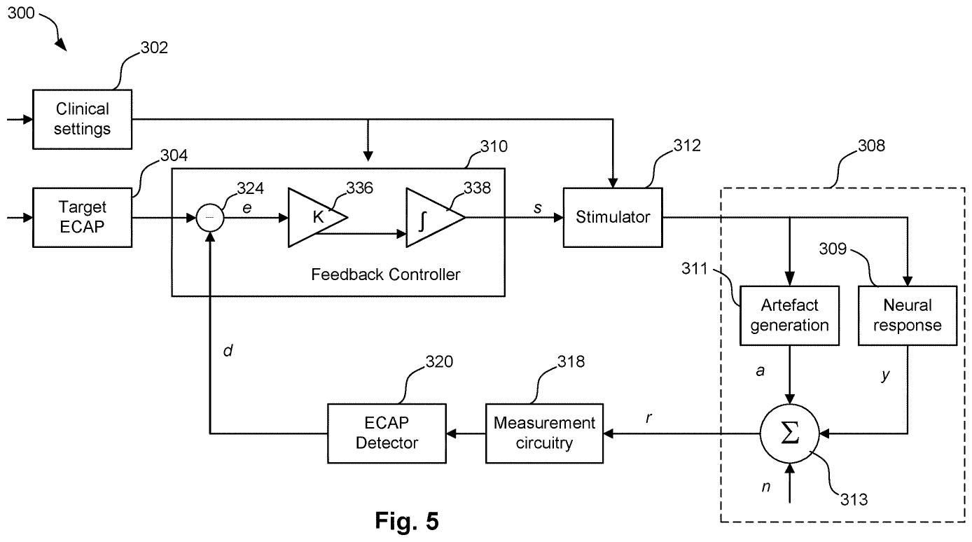

FIG. 5 is a schematic illustrating elements and inputs of a closed-loop neural stimulation system (CLNS) 300, according to one implementation of the present technology. The system 300 comprises a stimulator 312 which converts a stimulus intensity parameter (for example a stimulus current amplitude) s, in accordance with a set of predefined stimulus parameters, to a neural stimulus comprising a sequence of electrical pulses on the stimulus electrodes (not shown in FIG. 5). According to one implementation, the predefined stimulus parameters comprise the number and order of phases, the number of stimulus electrode poles, the pulse width, and the stimulus rate or frequency.

The generated stimulus crosses from the electrodes to the spinal cord, which is represented in FIG. 5 by the dashed box 308. The box 309 represents the evocation of a neural response y by the stimulus as described above. The box 311 represents the evocation of an artefact signal a, which is dependent on stimulus intensity and other stimulus parameters, as well as the electrical environment of the measurement electrode. Various sources of noise n, as well as the artefact a, may add to the evoked response y at the summing element 313 to form the sensed signal r, including electrical noise from external sources such as 50 Hz mains power; electrical disturbances produced by the body such as neural responses evoked not by the device but by other causes such as peripheral sensory input, EEG, EMG, and electrical noise from measurement circuitry 318.

The neural recruitment arising from the stimulus is affected by mechanical changes, including posture changes, walking, breathing, heartbeat and so on. Mechanical changes may cause impedance changes, or changes in the location and orientation of the nerve fibres relative to the electrode array(s). As described above, the intensity of the evoked response provides a measure of the recruitment of the fibres being stimulated. In general, the more intense the stimulus, the more recruitment and the more intense the evoked response. An evoked response typically has a maximum amplitude in the range of microvolts, whereas the voltage resulting from the stimulus applied to evoke the response is typically several volts.

Measurement circuitry 318, which may be identified with measurement circuitry 128, amplifies the sensed signal r (including evoked neural response, artefact, and noise) and samples the amplified sensed signal r to capture a “signal window” comprising a predetermined number of samples of the amplified sensed signal r. The ECAP detector 320 processes the signal window and outputs a measured neural response intensity d. A typical number of samples in a captured signal window is 60. In one implementation, the neural response intensity comprises an ECAP amplitude. The measured response intensity d is input into the feedback controller 310. The feedback controller 310 comprises a comparator 324 that compares the measured response intensity d to a target ECAP amplitude as set by the target ECAP controller 304 and provides an indication of the difference between the measured response intensity d and the target ECAP amplitude. This difference is the error value, e.

The feedback controller 310 calculates an adjusted stimulus intensity parameter, s, with the aim of maintaining a measured response intensity d equal to the target ECAP amplitude. Accordingly, the feedback controller 310 adjusts the stimulus intensity parameter s to minimise the error value, e. In one implementation, the controller 310 utilises a first order integrating function, using a gain element 336 and an integrator 338, in order to provide suitable adjustment to the stimulus intensity parameter s. According to such an implementation, the current stimulus intensity parameter s may be computed by the feedback controller 310 as

$$s = \int Kedt \qquad ( 2 )$$

where K is the gain of the gain element 336 (the controller gain). This relation may also be represented as

$$δ s = Ke \qquad ( 3 )$$

where δs is an adjustment to the current stimulus intensity parameter s.

A target ECAP amplitude is input to the comparator 324 via the target ECAP controller 304. In one embodiment, the target ECAP controller 304 provides an indication of a specific target ECAP amplitude. In another embodiment, the target ECAP controller 304 provides an indication to increase or to decrease the present target ECAP amplitude. The target ECAP controller 304 may comprise an input into the neural stimulus device, via which the patient or clinician can input a target ECAP amplitude, or indication thereof. The target ECAP controller 304 may comprise memory in which the target ECAP amplitude is stored, and from which the target ECAP amplitude is provided to the feedback controller 310.

A clinical settings controller 302 provides clinical settings to the system, including the gain K for the gain element 336 and the stimulation parameters for the stimulator 312. The clinical settings controller 302 may be configured to adjust the gain K of the gain element 336 to adapt the feedback loop to patient sensitivity. The clinical settings controller 302 may comprise an input into the neural stimulus device, via which the patient or clinician can adjust the clinical settings. The clinical settings controller 302 may comprise memory in which the clinical settings are stored, and are provided to components of the system 300.

In some implementations, two clocks (not shown) are used, being a stimulus clock operating at the stimulus frequency (e.g. 60 Hz) and a sample clock for sampling the sensed signal r (for example, operating at a sampling frequency of 10 kHz). As the ECAP detector 320 is linear, only the stimulus clock affects the dynamics of the CLNS 300. On the next stimulus clock cycle, the stimulator 312 outputs a stimulus in accordance with the adjusted stimulus intensity s. Accordingly, there is a delay of one stimulus clock cycle before the stimulus intensity is updated in light of the error value e.

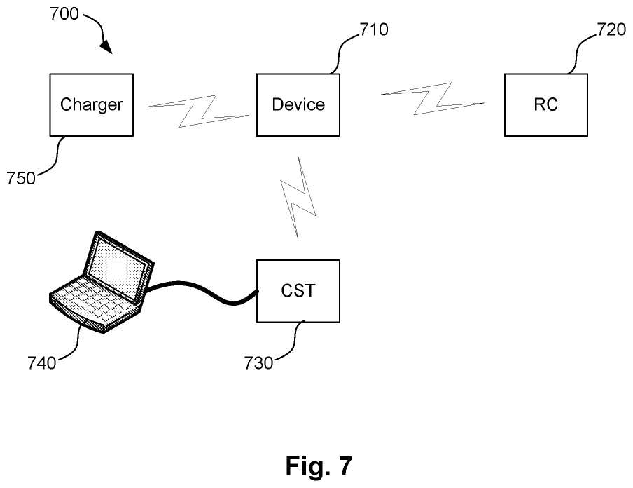

FIG. 7 is a block diagram of a neural stimulation system 700. The neural stimulation system 700 is centred on a neuromodulation device 710. In one example, the neuromodulation device 710 may be implemented as the stimulator 100 of FIG. 1, implanted within a patient (not shown). The neuromodulation device 710 is connected wirelessly to a remote controller (RC) 720. The remote controller 720 is a portable computing device that provides the patient with control of their stimulation in the home environment by allowing control of the functionality of the neuromodulation device 710, including one or more of the following functions: enabling or disabling stimulation; adjustment of stimulus intensity or target neural response intensity; and selection of a stimulation control program from the control programs stored on the neuromodulation device 710.

The charger 750 is configured to recharge a rechargeable power source of the neuromodulation device 710. The recharging is illustrated as wireless in FIG. 7 but may be wired in alternative implementations.

The neuromodulation device 710 is wirelessly connected to a Clinical System Transceiver (CST) 730. The wireless connection may be implemented as the transcutaneous communications channel 190 of FIG. 1. The CST 730 acts as an intermediary between the neuromodulation device 710 and the Clinical Interface (CI) 740, to which the CST 730 is connected. A wired connection is shown in FIG. 7, but in other implementations, the connection between the CST 730 and the CI 740 is wireless.

The CI 740 may be implemented as the external computing device 192 of FIG. 1. The CI 740 is configured to program the neuromodulation device 710 and recover data stored on the neuromodulation device 710. This configuration is achieved by program instructions collectively referred to as the Clinical Programming Application (CPA) and stored in an instruction memory of the CI 740.

The CPA makes use of a user interface (UI) of the CI 740. The UI may comprise a device for displaying information to the user (e.g. a display) and a device for receiving input from the user, such as a touchscreen, movable pointing device controlling a cursor (mouse), keyboard, joystick, touchpad, trackball etc. In the example of a touchscreen, the input device may be combined with the display. Alternatively, the UI of the CI 740 the input device(s) may be separate from the display.

### The Assisted Programming System

As mentioned above, obtaining patient feedback about their sensations is important during programming of closed-loop neural stimulation therapy, but mediation by trained clinical engineers is expensive and time-consuming. It would therefore be advantageous if patients could program their own implantable device themselves, or with some assistance from a clinician. However, interfaces for current programming systems are non-intuitive and generally unsuitable for direct use by patients because of their technical nature. There is therefore a need for a CPA to be as intuitive for non-technical users as possible while avoiding discomfort to the patient.

Implementations of an Assisted Programming System (APS) according to the present technology are generally configured to meet this need. In some implementations, the APS comprises two elements: the Assisted Programming Module (APM), which forms part of the CPA, and the Assisted Programming Firmware (APF), which forms part of the control programs 122 executed by the controller 116 of the electronics module 110. The data obtained from the patient is analysed by the APM to determine the parameters and settings for the neural stimulation therapy to be delivered by the stimulator 100. The APF is configured to complement the operation of the APM by responding to commands issued by the APM via the CST 730 to the stimulator 100 to deliver specified stimuli to the patient, and by returning, via the CST 730, measurements of neural responses to the delivered stimuli.

In other implementations, all the processing of the APS according to the present technology is done by the APF. In other words, the data obtained from the patient is not passed to the APM, but is analysed by the APF to determine the parameters and settings for the neural stimulation therapy to be delivered by the stimulator 100.

In implementations of the APS in which the APM analyses the data from the patient, the APS instructs the device 710 to capture and return signal windows to the CI 740 via the CST 730. In such implementations, the device 710 captures the signal windows using the measurement circuit 128 and bypasses the ECAP detector 320, storing the data representing the raw signal windows temporarily in memory 118 before transmitting the data representing the captured signal windows to the APS for analysis.

Following the processing, the APS may load the determined program onto the device 710 to govern subsequent neural stimulation therapy. In one implementation, the program comprises clinical settings 121, also referred to as therapy parameters, that are input to the neuromodulation device by, or stored in, the clinical settings controller 302. The patient may subsequently control the device 710 to deliver the therapy according to the determined program using the remote controller 720 as described above. The determined program may also, or alternatively, be loaded into the CPA for validation and modification.

### Noise Departure Detector (NDD)

In some implementations, the APS according to the present technology comprises a noise departure detector (NDD). The NDD is a statistical detector of the presence of an ECAP in a signal window. The operation of the NDD on a signal window is preferably preceded by an “artefact scrubber” which removes artefact from the signal window. On such artefact scrubber is disclosed by the present applicant in International Patent Publication no. WO2020/124135, the entire contents of which are herein incorporated by reference. The NDD works by detecting a statistically unusual difference from the expected noise present in a signal window. The extent of the difference is indicative of the likelihood of an ECAP in the signal window.

The calibration of an NDD instance corresponding to a measurement electrode configuration (MEC) may be carried out on one or more signal windows that are known not to contain evoked neural responses. In one implementation, such signal windows are “zero current” signal windows which are captured from intervals during which no stimulus is being delivered, and which have preferably been scrubbed for artefact, and may therefore be treated as comprising only noise. The calibration comprises forming estimates of parameters of a predetermined “noise model” (statistical distribution) from the samples in the one or more “zero current” signal windows. In one implementation, the noise model is Gaussian and the parameters are the mean \tilde{μ} and standard deviation \hat{σ} of the samples.

Once calibrated, an NDD instance may be applied to a signal window by counting the number \tilde{k} of outliers in the signal window, i.e. the number of samples in the signal window that depart significantly from the noise model. For a Gaussian noise model, the NDD counts the number \tilde{k} of samples that differ from the mean estimate \tilde{μ} by more than n times the standard deviation estimate \hat{σ}, where n is a small integer. The number \tilde{k} of outliers is compared to the number \hat{k} of such outliers that would be expected to occur if the signal window consisted solely of noise with mean \tilde{μ} and standard deviation \hat{σ}. The difference between \tilde{k} and \hat{k} is divided by the number of samples N in the signal window to obtain a metric r that quantifies the ratio of outliers present in a signal window relative to the expected ratio of outliers in a signal window that obeys the noise model.

It may be shown that for Gaussian noise model, the NDD may estimate the metric r as

$$r = \frac{\tilde{k}}{N} - 2 Φ ( \frac{n}{2} ) \qquad ( 4 )$$

where Φ is the standard normal cumulative distribution function.

A negative or zero value of the metric r indicates a signal window consistent with the noise model, whereas a positive value of r indicates a departure from the noise model. Such a departure is deemed to be due to the presence of an ECAP in the signal window.

In one implementation of the NDD, n is set to 3. Smaller values of n make the NDD more sensitive, indicating a departure from noise more readily and increasing the rate of Type I errors (false positives). Conversely, high values for n necessitate large outliers before r will indicate a noise departure, increasing the rate of Type II errors (false negatives).

In one implementation of the NDD, a sigmoid function may be applied to the raw metric r to map the metric r to a quality indicator Q_{NDD} in the interval [0, 1]:

$${Q}_{NDD} = \frac{1}{1 + exp ( - γ r )} \qquad ( 5 )$$

where γ is a parameter that balances the Type I and Type II errors. The quality indicator Q_{NDD} has a natural interpretation: Q_{NDD}<0.5 corresponds to r≤0 and indicates that the signal window is most likely noise. Conversely, Q_{NDD}>0.5 indicates a departure from the noise model that is deemed to represent an ECAP. In one implementation, γ is set to 50.

In one implementation, the NDD may be applied to multiple signal windows after they have been averaged together to improve the signal-to-noise ratio. In one such implementation, the number of averaged signal windows is eight. In such implementations, the parameters of the noise model may be adjusted depending on the number of signal windows that are averaged. In the Gaussian noise model, the standard deviation \hat{σ} should be divided by the square root of the number of averaged signal windows.

### Estimating Key Patient Response Parameters

One specific aim of the APS according to the present technology is to estimate the key parameters of the patient's response to stimulus, as represented by the activation plot illustrated in FIG. 4a in idealised form. The key parameters include the ECAP threshold T and the patient sensitivity S. In general, these key parameters vary depending on the SEC in use and the posture of the patient. As it may not be known which SEC will provide the optimum pain relief for the patient, it is useful to estimate the key parameters for a number of candidate SECs that may be selected for use by the patient as part of their stimulation program. However, as mentioned above, discomfort thresholds vary widely between patients, between postures for a single patient, and between SECs for a given patient in a given posture. It is difficult to know in advance where a given patient's discomfort threshold is for a given SEC in a given posture. The result is that a test stimulus of an intensity that is comfortable for one patient may provoke acute discomfort for another patient, or for the same patient in a different posture, or for the same patient in the same posture when applied at a different SEC. This means the measurement of the intensity of patients' neural responses across the therapeutic range of stimulus intensity at a particular SEC, as ideally would be performed to obtain the activation plot for that SEC, is liable to cause discomfort if carried out without prior knowledge of the therapeutic range or real-time patient feedback. One alternative to avoid discomfort is to slowly increment stimulus intensity and solicit patient feedback as to their level of comfort, but this is time-consuming particularly if many SECs and postures are to be measured.

The APS according to the present technology may use predetermined values of certain stimulus parameters. In one implementation, those stimulus parameters and values are:
Pulse width: 240 microseconds
Inter-phase gap: 200 microseconds
Pulse shape: triphasic, with anodic phase first

Another stimulus parameter, stimulus frequency, may be selected as described below. The choice of stimulus frequency fₛ sets the inter-stimulus interval ISI as follows:

$$ISI = \frac{1}{{f}_{s}} - L \qquad ( 6 )$$

where L is the stimulus pulse width, which for example is equal to 1.12 ms according to the stimulus parameter values listed above.

Yet another stimulus parameter, stimulus intensity, may vary as described below.

In one implementation of the present technology, four candidate stimulus electrode configurations (SECs) are defined. Each SEC is tripolar, comprising a stimulus electrode that acts primarily as a cathode, sinking stimulus current, with the two neighbouring return electrodes on either side of the stimulus electrode acting primarily as anodes, sourcing return currents. Tripolar stimulus electrode configurations are described in more detail in International Patent Publication no. WO2017/219096 by the present applicant, the entire contents of which are herein incorporated by reference.

According to this implementation, the electrode array 150 consists of two leads implanted approximately symmetrically to left and right (as viewed from behind the patient) of the patient's midline, as illustrated in FIG. 1. In one implementation, each lead comprises twelve contacts (electrodes), numbered such that a contact index of zero is the topmost (rostral) contact of a lead and contact index 11 is the bottom-most (caudal) contact of a lead. The stimulus electrodes in each of the four candidate SECs are defined as follows: top left (contact index 1, left lead), top right (contact index 1, right lead), bottom left (contact index 10, left lead) and bottom right (contact index 10, right lead). In other implementations with a different number of contacts in each lead, the bottom left and bottom right stimulus electrodes are defined to be the second-most caudal contact on the respective leads.

For each SEC, the APS defines at least one measurement electrode configuration (MEC). A measurement electrode configuration comprises two electrodes for differential ECAP recording, as illustrated in FIG. 3. The measurement electrode connected to the positive terminal of the measurement circuitry 318 is referred to as the recording electrode, while the measurement electrode connected to the negative terminal of the measurement circuitry 318 is referred to as the reference electrode. In one implementation, an MEC for a tripolar SEC comprises a recording electrode separated by four contacts from the central electrode of the tripole, and a reference electrode separated by a further two electrodes from the recording electrode.

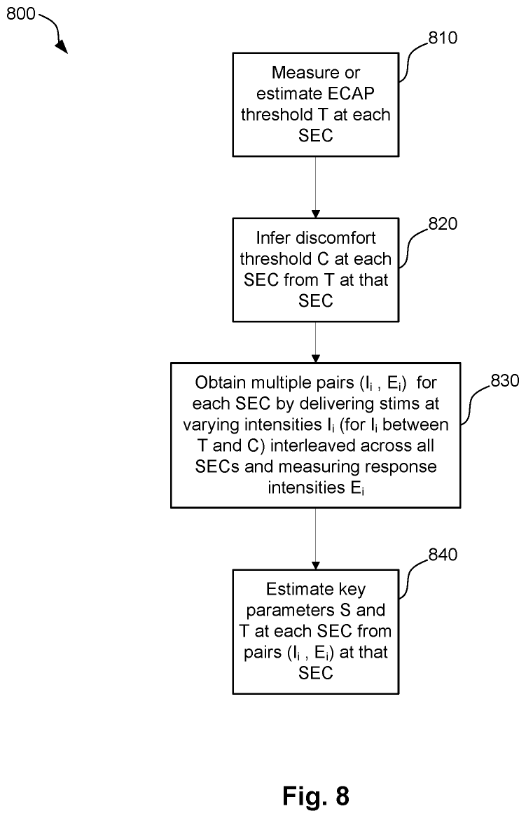

FIG. 8 is a flow chart illustrating a method 800 of estimating key parameters of the patient's response to stimulus at multiple SECs simultaneously. The method 800 may form part of the APS as described above.

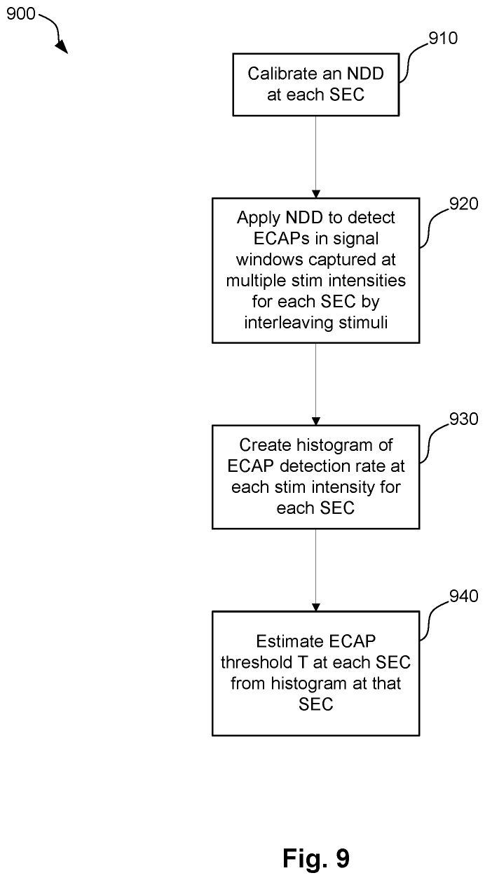

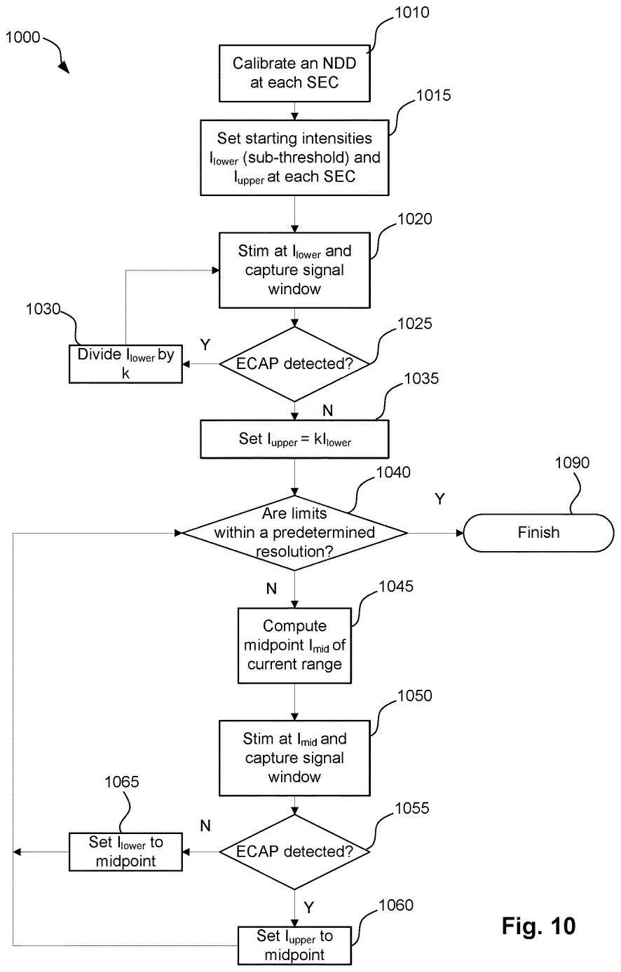

The method 800 starts at step 810, which measures or estimates the ECAP threshold T at each SEC. Step 810 may use prior patient data comprising ECAP thresholds for many patients, together with their characteristics, to estimate the ECAP threshold for each SEC. In one such implementation, ECAP thresholds from patients with similar characteristics to the current patient 108, for example the absolute position of the SEC in relation to the spinal cord, are retrieved from the patient data and a representative ECAP threshold value is extracted from the retrieved ECAP thresholds. Alternatively, some stimulus and response measurements may be made to estimate the ECAP threshold for each SEC. Two such implementations are described below with reference to FIGS. 9 and 10. If any SEC has multiple defined MECs, step 810 may use any of the MECs that are defined for that SEC through which to measure the responses, as the ECAP threshold for an SEC is generally insensitive to the MEC used for that SEC.

Step 820 then infers a discomfort threshold Max at each SEC from the ECAP threshold T at that SEC. In one implementation, step 820 uses a linear prediction model:

$$Max = m \cdot T \qquad ( 7 )$$

where m is a correlation parameter that may be derived from patient data comprising many values of ECAP threshold T and corresponding values of discomfort threshold Max at a given SEC. In one implementation, m takes a value between 1.0 and 2.0. In another implementation, m takes a value between 1.1 and 1.6. In one implementation, m takes a value between 1.25 and 1.5.

Step 830 then obtains a set of pairs {(Iᵢ, Eᵢ), i=1, . . . , N}, where N>1, for each SEC, where Iiis an intensity parameter of a delivered stimulus (e.g. stimulus current pulse amplitude) and Eᵢ is the measured intensity of the neural response evoked by the stimulus, e.g. an ECAP peak-to-peak amplitude. Step 830 instructs the device 710 to deliver stimuli of varying intensities Iᵢ via the SECs in a sequence and to return the measured response intensity Eᵢ, where each stimulus intensity Iᵢ is between the ECAP threshold T and the discomfort threshold Max for the corresponding SEC, as determined at steps 810 and 820 respectively. The delivered stimuli are separated in time by the inter-stimulus interval ISI. The delivery cycle is then repeated. This scheme is referred to as interleaved stimulation or the “shotgun approach”. The order in which the SECs are stimulated from may be the same in each cycle, or may be permuted, e.g. stochastically permuted, between cycles. In one implementation in which the sequence order is fixed between cycles, the order is configured to maximize the geometric distance between consecutive SECs such that the time elapsed between stimulus delivery at an SEC and stimulus delivery at a neighbouring SEC is greater than the refractory period. For example, for the four-SEC arrangement described above the order may be [top left], [bottom left], [top right], [bottom right].

In an implementation in which the sequence order of SECs varies, Markov sampling may be used to select the next SEC in the sequence. In Markov sampling, the probability of the next SEC selected is a function of the history of SECs previously selected, as specified in a transition probability matrix. For instance, the transition probability matrix may be configured such that if the recently selected SECs have predominantly been on the left lead, then the probability of selecting an SEC on the right lead is high, while the probability of selecting an SEC on the left lead is low. In one such implementation, the transition probability matrix may change depending on whether an ECAP was detected at the most recently selected SEC.

The intensities Iᵢ may be chosen stochastically between the ECAP threshold T and the discomfort threshold Max for the corresponding SEC. In one such implementation, the intensities Iᵢ may be sampled from a uniform distribution between the ECAP threshold T and the discomfort threshold Max for the corresponding SEC. In other such implementations, the distributions from which the intensities Iᵢ are sampled may be normal distributions whose parameters at a given SEC depend dynamically on measurements from preceding SECs, particularly neighbouring SECs. In one such implementation, the mean and standard deviation of the distribution at each SEC is set to a default. After stimulating at one SEC, labelled SEC1, the mean of the distribution at SEC2, which neighbours SEC1, is set to half of the default mean if an ECAP was detected on SEC1 or double the default mean if an ECAP was not detected on SEC1. The increase in standard deviation of the distribution may be a function of the geometric distance between the SECs to reflect the increased uncertainty in stimulation parameters for SECs that are further away.

The use of the shotgun approach in step 830 means that if the occasional stimulus intensity is above the actual discomfort threshold for a particular SEC (due to incorrect inference of the discomfort threshold at that SEC at step 820), the patient will feel little to no discomfort, as the effect of the uncomfortably intense stimulus is psychophysically masked by the adjacent, comfortable stimuli at different SECs. The potential discomfort of step 830 is therefore minimised compared to a method by which the key parameters are estimated at each SEC separately in time from the estimation of the key parameters at other SECs.

Step 840 then uses the set of pairs {(Iᵢ, Eᵢ), i=1, . . . , N} for each SEC to estimate the key parameters of the activation plot at that SEC. Step 840 is described in more detail below. The method 800 then concludes.

If any SEC has multiple defined MECs, step 830 may obtain a set of pairs {(Iᵢ, Eᵢ)} for each MEC that is defined for that SEC. This may be done for a stimulus of intensity Iᵢ by making a measurement of response intensity Eᵢ at each of the multiple MECs defined for that SEC. Step 840 may then use the pairs {(Iᵢ, Eᵢ)} for each SEC/MEC pair to estimate the key parameters of the activation plot at that SEC/MEC pair.

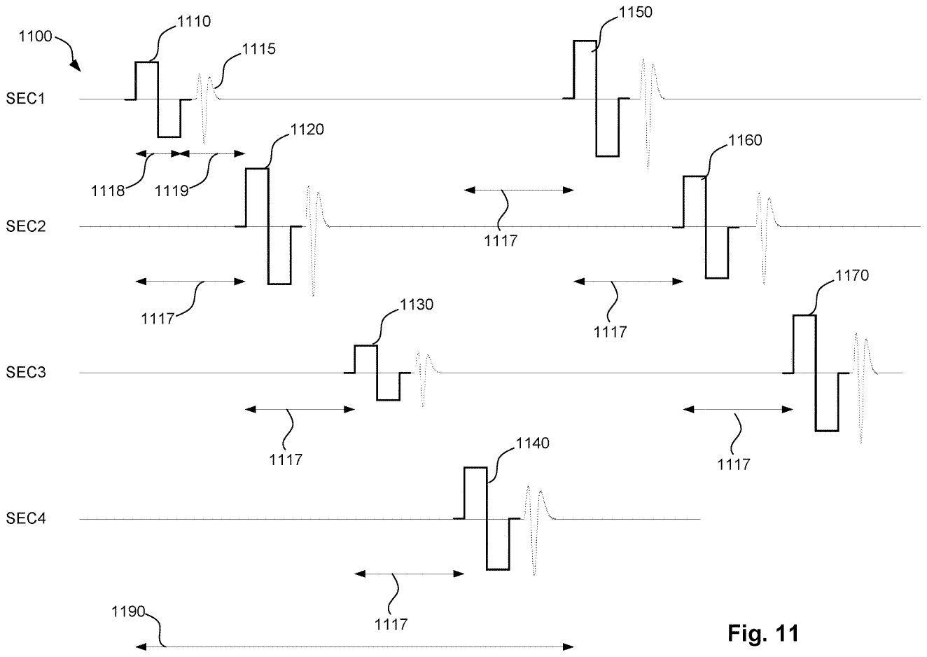

FIG. 11 contains an illustration of a timing sequence 1100 of stimulus pulses according to one implementation of the present technology. In the implementation illustrated in FIG. 11, there are four SECs, each with a corresponding timeline, labelled SEC1, SEC2, SEC3, and SEC4. In the timing sequence 1100, a (biphasic) stimulus pulse 1110 is delivered via SEC1, followed by a stimulus pulse 1120 via SEC2, a stimulus pulse 1130 via SEC3, and a stimulus pulse 1140 via SEC4. The stimulus pulses 1110, 1120, 1130, and 1140 make up one cycle of the timing sequence 1100. Each stimulus pulse has a constant pulse width L 1118, which is for example equal to 1.12 ms according to the stimulus parameter values listed above. The stimulus period Tₛ 1117 is the reciprocal of the stimulus frequency fs and, according to Equation (6), is equal to the stimulus pulse width L plus the inter-stimulus interval ISI 1119. The implementation illustrated by the timing sequence 1100 is one in which the order of SECs is constant between cycles, as shown by the fact that the stimulus pulse 1140 is followed by a stimulus pulse 1150 delivered via SEC1, which in turn is followed by a stimulus pulse 1160 delivered via SEC2, and then a stimulus pulse 1170 delivered via SEC3. The stimulus pulses at an SEC vary in amplitude from cycle to cycle, as illustrated by the stimulus pulse 1150 at SEC1 being of greater intensity (amplitude) than the stimulus pulse 1110 delivered via SEC1 in the first cycle.

Each stimulus pulse is followed by an evoked neural response, e.g. the stimulus pulse 1110 is followed by an ECAP 1115.

The stimulus frequency fₛ may be chosen by the APS such that the neural response characteristic measurement is not significantly affected by the tissue adjacent each SEC either being in the refractory period of the previously delivered stimulus pulse at that SEC, or being in the depolarising after-potential of the response. In one such implementation, the effective inter-stimulus interval at each SEC is chosen to be longer than the refractory period plus the depolarising after-potential period. Labelling that total period as D, and the number of SECs as M, to meet this constraint the stimulus frequency fₛ may be chosen as

$${f}_{s} < \frac{M}{D + L} \qquad ( 8 )$$

The effective inter-stimulus interval at each SEC is MTₛ-L. The effective stimulus frequency at each SEC is the chosen stimulus frequency fₛ divided by M, or equivalently, the effective stimulus period at each SEC is the chosen stimulus period Tₛ multiplied by M. For example, in the timing sequence 1100, the effective stimulus period at SEC1 is labelled as 1190. If the sequence order of SECs in step 830 is variable rather than fixed, these effective values are accurate on the average, though the actual values will vary between stimuli at that SEC. The inter-stimulus interval ISI may be computed from the chosen stimulus frequency fₛ using equation (6).

Step 840 of the method 800 uses the set of pairs {(li, Eᵢ), i=1, . . . , N} for each SEC to estimate the key parameters of the activation plot at that SEC. In one implementation of step 840, a straight line is fit to the pairs (Iᵢ, Eᵢ), for example using conventional linear regression. As modelled by Equation (1), the slope and x-intercept of the fitted line are the sensitivity S and ECAP threshold T for that SEC.

In an alternative implementation, step 840 uses a process called the activation plot builder to fit a model referred to as the Logistic Growth Curve (LGC) to the pairs (Iᵢ, Eᵢ) for each SEC. In one implementation, the LGC model is a four-parameter function of stimulus intensity I:

$$E ( I ) = A + \frac{K - A}{1 + exp ( - B ( I - M ) )} \qquad ( 9 )$$

where the four parameters are:
A, the minimum value (the detected ECAP amplitude in the absence of stimulation)
K, the maximum value (the detected ECAP amplitude at which saturation occurs, i.e. increases in stimulus intensity do not increase the detected ECAP amplitude)
M, the current amplitude at the midpoint between A and K
B, the steepness of the LGC, which is proportional to the gradient at the midpoint between A and K.

In other implementations, fewer parameters may be used for the LGC model, for example an LGC model in which the minimum value A is identically zero.

To fit the LGC, the parameters A, K, M, and B may be initialised to sensible starting points A₀, K₀, M₀, and B₀. In one implementation, these values may be set to:
A₀: the mean of the ECAP amplitudes obtained from the lowest few stimulus current amplitudes.
K₀: the mean of the ECAP amplitudes obtained from the highest few stimulus current amplitudes.
M₀: the stimulus current amplitude at the midpoint between A and K
B₀: may be calculated from the gradient m at the midpoint, obtained from local linear regression of pairs (Iᵢ, Eᵢ) acquired near the midpoint, as B₀=m*4/(K₀−A₀).

An optimisation algorithm such as Trust Region Reflective (TRF) may then be used to optimise the four parameters A, K, M, and B from their starting points A₀, K₀, M₀, and B₀.

The fitted LGC may be used to estimate the ECAP threshold T. In one implementation, a line is constructed through the midpoint M of the fitted LGC with slope B. The ECAP threshold Iₜₕᵣₑₛₕ may be estimated as the stimulus current amplitude s at which the constructed line intersects the minimum value A. It may be shown that the resulting ECAP threshold Tis given by

$$T = M - \frac{2}{B} \qquad ( 10 )$$

The fitted LGC may be used to estimate the patient sensitivity S. In one implementation, the patient sensitivity S is the slope of the fitted LGC at its midpoint M, which may be computed from the steepness B as follows:

$$S = \frac{B}{4} ( K - A ) \qquad ( 11 )$$

The measured sensitivity S at each SEC may be adjusted for the difference between the effective stimulus frequency of the shotgun approach at that SEC (which is fₛ divided by M), and the ultimate therapeutic stimulus frequency Fₛ to be used at that SEC. In one implementation, this adjustment is based on a logarithmic dependence of ECAP amplitude on stimulus frequency as disclosed in Gmel 0. Since sensitivity is a function of ECAP amplitude, a similar logarithmic dependence of sensitivity on stimulus frequency may be assumed.

Once the key patient response parameters have been estimated for each SEC, e.g. by the method 800, they may be used by the APS to determine clinical settings for the CLNS system 300. In one implementation, the measured sensitivity S (possibly adjusted for effective stimulus frequency) may be used to set the gain K of the gain element 336 for the corresponding SEC. International Patent Publication no. WO2016/090436, the contents of which are incorporated herein by reference, describes how the controller gain K may be set based on the measured sensitivity S. In another implementation, the initial ECAP target may be set for an SEC based on the measured ECAP threshold T and the measured sensitivity S at that SEC.

FIG. 9 is a flow chart illustrating a method 900 of using the NDD to estimate the ECAP threshold T at each SEC. The method 900 is one implementation of step 810 of the method 800 described above. The method 900 starts at step 910, which creates and calibrates an instance of the NDD at each SEC as described above. Step 920 delivers stimuli of varying intensities at each SEC using the shotgun approach as described above, and uses the calibrated NDD at the corresponding SEC to detect ECAPs in the captured signal windows as described above. The stimulus intensities may be varied stochastically around a rough estimate of the ECAP threshold at each SEC. In one implementation, the rough estimate is 5 mA. In another implementation, step 920 may use prior patient data comprising ECAP thresholds for many patients, together with their characteristics, to provide the rough estimate of the ECAP threshold for each SEC. In one such implementation, ECAP thresholds from patients with similar characteristics to the current patient 108, for example the absolute position of the SEC in relation to the spinal cord, are retrieved from the patient data and a representative ECAP threshold value is extracted from the retrieved ECAP thresholds.

As in step 830, the use of the shotgun approach in step 920 means that if the occasional stimulus intensity is above the actual discomfort threshold for a particular SEC, the patient will feel little to no discomfort, as the effect of the uncomfortably intense stimulus is psychophysically masked by the adjacent, comfortable stimuli at different SECs.

Step 930 creates a histogram of stimulus intensities at which ECAPs were detected for each SEC. The value of the histogram for a stimulus intensity bin is the number of signal windows found to have contained an ECAP whose stimulus intensity lies within that intensity bin. The histogram may be normalised by dividing the value in each bin by the total number of signal windows whose intensity lies within that intensity bin. The value of the normalised histogram for a stimulus intensity bin is the ECAP detection rate at that stimulus intensity.

Finally, step 940 uses the normalised histogram for each SEC to estimate the ECAP threshold at that SEC. In one implementation, step 940 interpolates the normalised histogram values to find the intensity at which the ECAP detection rate is 50%.

FIG. 10 is a flow chart illustrating a method 1000 of using the NDD to estimate the ECAP threshold T at each SEC. The method 1000 is one implementation of step 810 of the method 800. The method 1000 carries out a binary search at each SEC, interleaved with the binary searches at all other SECs, to estimate the ECAP threshold T at that SEC. The method 1000 starts at step 1010, which creates and calibrates an instance of the NDD at each SEC as described above. Step 1015 sets a starting lower stimulus intensity limit I_{lower} at each SEC. The lower stimulus intensity limit I_{lower} should be set lower than a rough estimate of the ECAP threshold at each SEC. In one implementation, the rough estimate is 5 mA. In another implementation, step 1015 may use prior patient data comprising ECAP thresholds for many patients, together with their characteristics, to provide the rough estimate of the ECAP threshold for each SEC. In one such implementation, ECAP thresholds from patients with similar characteristics to the current patient 108, for example the absolute position of the SEC in relation to the spinal cord, are retrieved from the patient data and a representative ECAP threshold value is extracted from the retrieved ECAP thresholds.

The following steps 1020 to 1090 are carried out across all SECs using the shotgun approach, applying stimuli and updating the lower and upper stimulus intensity limits I_{lower} and Iᵤₚₚₑᵣ at each SEC based on the results. Step 1020 delivers a stimulus at the lower stimulus intensity limit I_{lower} and captures the resulting signal window. Step 1025 applies the NDD to detect an ECAP in the captured signal window. If an ECAP is detected (“Y”), step 1030 divides I_{lower} by a constant k that is greater than 1 to reduce its value, and the method returns to step 1020. Once I_{lower} is small enough that no ECAP is detected (“N”), step 1035 multiplies I_{lower} by k to obtain the upper stimulus intensity limit Iᵤₚₚₑᵣ (equal to the previous value of I_{lower} at which an ECAP was detected). The method 1000 now has values I_{lower} and Iᵤₚₚₑᵣ at each SEC as lower and upper limits of a range of stimulus intensities that includes the ECAP threshold at that SEC.

Step 1040 checks whether the difference between the limits I_{lower} and Iᵤₚₚₑᵣ at each SEC is within a predetermined resolution. In one implementation, the predetermined resolution is that of the pulse generator 124. If so (“Y”), the method 1000 ends at step 1090 by setting the ECAP threshold to the lower limit I_{lower}.

If not (“N”), step 1045 then finds the midpoint Imid of the current range, e.g. by averaging the limits I_{lower} and Iᵤₚₚₑᵣ. Step 1050 delivers a stimulus at the stimulus intensity Imid and captures the resulting signal window. Step 1055 applies the NDD to detect an ECAP in the captured signal window. If an ECAP is detected (“Y”), step 1060 sets the upper limit Iᵤₚₚₑᵣ to Imid, and the method 1000 returns to step 1040 to continue the binary search. If no ECAP is detected (“N”), step 1065 sets the lower limit I_{lower} to Imid, and the method 1000 returns to step 1040 to continue the binary search. Other stopping criteria may be applied at step 1045, for example a fixed number of iterations through the loop over steps 1040 to 1065. The method 1000 converges exponentially to the stimulus intensity nearest which ECAPs are first evoked for each SEC.

As in step 830, the use of the shotgun approach in steps 1020 to 1090 means that if the occasional stimulus intensity is above the actual discomfort threshold for a particular SEC, the patient will feel little to no discomfort, as the effect of the uncomfortably intense stimulus is psychophysically masked by the adjacent, comfortable stimuli at different SECs.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not limiting or restrictive.

### REFERENCES

[1] Gmel, G. et al. (2021 January). The Effect of Spinal Cord Stimulation Frequency on the Neural Response and Perceived Sensation in Patients with Chronic Pain. Frontiers in Neuroscience, 15, 1-7.

LABEL LIST

| | |
|---|---|
| stimulator | 100 |
| patient | 108 |
| electronics module | 110 |
| battery | 112 |
| telemetry module | 114 |
| controller | 116 |
| memory | 118 |
| clinical data | 120 |
| clinical settings | 121 |
| control programs | 122 |
| pulse generator | 124 |
| electrode selection module | 126 |
| measurement circuitry | 128 |
| system ground | 130 |
| electrode array | 150 |
| biphasic stimulus pulse | 160 |
| ECAP | 170 |
| nerve | 180 |
| communications channel | 190 |
| external device | 192 |
| CLNS system | 300 |
| clinical settings controller | 302 |
| target ECAP controller | 304 |
| box | 308 |
| box | 309 |
| feedback controller | 310 |
| box | 311 |
| stimulator | 312 |
| element | 313 |
| measurement circuitry | 318 |
| ECAP detector | 320 |
| comparator | 324 |
| gain element | 336 |
| integrator | 338 |
| activation plot | 402 |
| ECAP threshold | 404 |
| discomfort threshold | 408 |
| perception threshold | 410 |
| therapeutic range | 412 |
| activation plot | 502 |
| activation plot | 504 |
| activation plot | 506 |
| ECAP threshold | 508 |
| ECAP threshold | 510 |
| ECAP threshold | 512 |
| ECAP target | 520 |
| ECAP | 600 |
| neural stimulation system | 700 |
| neuromodulation device | 710 |
| remote controller | 720 |
| clinical system transceiver | 730 |
| clinical interface | 740 |
| charger | 750 |
| method | 800 |
| step | 810 |
| step | 820 |
| step | 830 |
| step | 840 |
| method | 900 |
| step | 910 |
| step | 920 |
| step | 930 |
| step | 940 |
| method | 1000 |
| step | 1010 |
| step | 1015 |
| step | 1020 |
| step | 1025 |
| step | 1040 |
| step | 1045 |
| step | 1050 |
| step | 1055 |
| step | 1065 |
| step | 1090 |
| timing sequence | 1100 |
| stimulus pulse | 1110 |
| ECAP | 1115 |
| stimulus period Ts | 1117 |
| constant pulse width L | 1118 |
| inter - stimulus interval ISI | 1119 |
| stimulus pulse | 1120 |
| stimulus pulse | 1130 |
| stimulus pulse | 1140 |
| stimulus pulse | 1150 |
| stimulus pulse | 1160 |
| stimulus pulse | 1170 |
| effective stimulus period | 1190 |

1. A neural stimulation system comprising:
a neural stimulation device for controllably delivering a neural stimulus, the neural stimulation device comprising:
a plurality of implantable electrodes including one or more stimulus electrodes and one or more sense electrodes, wherein a stimulus electrode configuration comprises at least one stimulus electrode acting as an anode and at least one stimulus electrode acting as a cathode;
a stimulus source configured to deliver neural stimuli via a stimulus electrode configuration to a neural pathway of a patient;
measurement circuitry configured to capture signal windows sensed at a sense electrode of the one or more sense electrodes in response to respective neural stimuli; and
a control unit configured to control the stimulus source to deliver each neural stimulus according to a stimulus intensity parameter; and
a processor configured to:
instruct the control unit to control the stimulus source to sequentially deliver a plurality of neural stimuli via respective stimulus electrode configurations of a plurality of stimulus electrode configurations according to respective values of the stimulus intensity parameter, wherein the value of the stimulus intensity parameter at each stimulus electrode configuration is different from a preceding value of the stimulus intensity parameter at that stimulus electrode configuration;
receive a captured signal window corresponding to each delivered neural stimulus;
measure an intensity of an evoked neural response in each captured signal window, thereby forming a plurality of (stimulus intensity parameter, response intensity) pairs for each stimulus electrode configuration; and
estimate one or more key parameters of an activation plot at each stimulus electrode configuration, using the plurality of (stimulus intensity parameter, response intensity) pairs for each stimulus electrode configuration.

2. The neural stimulation system of claim 1, wherein the processor is configured to:
instruct the control unit to control the stimulus source to sequentially deliver a first plurality of neural stimuli via a first stimulus electrode configuration according to respective values of the stimulus intensity parameter; and
instruct the control unit to control the stimulus source to sequentially deliver, interleaved with the first plurality of neural stimuli, a second plurality of neural stimuli via a second stimulus electrode configuration according to respective values of the stimulus intensity parameter.

3. The neural stimulation system of claim 1, wherein the processor is configured to deliver the stimuli from the respective stimulus electrode configurations in an order which is the same when repeated.

4. The neural stimulation system of claim 3, wherein the order is configured to maximize a geometric distance between consecutively used stimulus electrode configurations.

5. The neural stimulation system of claim 1, wherein the processor is configured to deliver the stimuli from the respective stimulus electrode configurations in an order which is permuted when repeated.

6. The neural stimulation system of claim 5, wherein the processor is configured to use Markov sampling to permute the order.

7. The neural stimulation system of claim 6, wherein the processor is configured to change a transition probability matrix of the Markov sampling, depending on whether a preceding response intensity was zero.

8. The neural stimulation system of claim 1, wherein the processor is configured to choose the value of the stimulus intensity parameter between an ECAP threshold T and a discomfort threshold Max for the corresponding stimulus electrode configuration.

9. The neural stimulation system of claim 8, wherein the processor is configured to sample the value of the stimulus intensity parameter from a uniform distribution between the ECAP threshold T and the discomfort threshold Max for the corresponding stimulus electrode configuration.

10. The neural stimulation system of claim 8, wherein the processor is configured to sample the value of the stimulus intensity parameter from respective normal distributions each having parameters which, at a respective stimulus electrode configuration, depend dynamically on measurements from one or more preceding stimulus electrode configurations.

11. The neural stimulation system of claim 10, wherein a mean and a standard deviation of the respective normal distribution at each stimulus electrode configuration is initially set to a default, and wherein after stimulating at a preceding stimulus electrode configuration the mean of the distribution at a neighbouring stimulus electrode configuration is reduced if a non-zero response intensity was detected from the preceding stimulus electrode configuration or is increased if a zero response intensity was detected on the preceding stimulus electrode configuration.

12. The neural stimulation system of claim 10, wherein after stimulating at the preceding stimulus electrode configuration, the standard deviation of the neighbouring stimulus electrode configuration distribution is altered by an amount which is a function of the geometric distance between the stimulus electrode configurations.

13. The neural stimulation system of claim 8, wherein the processor is further configured to estimate the discomfort threshold at each stimulus electrode configuration from the ECAP threshold at that stimulus electrode configuration.

14. The neural stimulation system of claim 13, wherein the processor is configured to estimate the discomfort threshold at each stimulus electrode configuration by applying a linear model to the ECAP threshold at that stimulus electrode configuration.

15. The neural stimulation system of claim 8, wherein the processor is configured to estimate the ECAP threshold at each stimulus electrode configuration.

16. The neural stimulation system of claim 15, wherein the processor is configured to estimate the ECAP threshold at a stimulus electrode configuration by applying a noise departure detector to detect ECAPs in captured signal windows corresponding to multiple neural stimuli of different stimulus intensity parameters delivered via the stimulus electrode configuration.

17. The neural stimulation system of claim 1, wherein the processor is configured to estimate the one or more key parameters at each stimulus electrode configuration by fitting an activation plot model to the plurality of (stimulus intensity parameter, response intensity) pairs for that stimulus electrode configuration.

18. The neural stimulation system of claim 17, wherein the activation plot model is a straight line, and one of the one or more key parameters is a slope of the fitted straight line.

19. The neural stimulation system of claim 17, wherein the activation plot model is a straight line, and one of the one or more key parameters is an intercept of the fitted straight line.

20. The neural stimulation system of claim 17, wherein the activation plot model is a logistic growth curve, and one of the one or more key parameters is a slope of the fitted logistic growth curve at its midpoint.

21. The neural stimulation system of claim 1, wherein the processor is part of the neural stimulation device.

22. The neural stimulation system of claim 1, further comprising an external computing device in communication with the neural stimulation device.

23. The neural stimulation system of claim 22, wherein the processor is part of the external computing device.

24. An automated method of estimating one or more key parameters of neural responses evoked by neural stimuli delivered to a patient, the method comprising:
sequentially delivering a plurality of neural stimuli via respective stimulus electrode configurations of a plurality of stimulus electrode configurations according to respective values of a stimulus intensity parameter, wherein the value of the stimulus intensity parameter at each stimulus electrode configuration is different from a preceding value of the stimulus intensity parameter at that stimulus electrode configuration;
receiving a captured signal window corresponding to each delivered neural stimulus;
measuring an intensity of an evoked neural response in each captured signal window, thereby forming a plurality of (stimulus intensity parameter, response intensity) pairs for each stimulus electrode configuration; and
estimating one or more key parameters of an activation plot at each stimulus electrode configuration, using the plurality of (stimulus intensity parameter, response intensity) pairs for each stimulus electrode configuration.

25. The method of claim 24, wherein the sequentially delivering comprises:
sequentially delivering a first plurality of neural stimuli via a first stimulus electrode configuration according to respective values of the stimulus intensity parameter; and
sequentially delivering, interleaved with the first plurality of neural stimuli, a second plurality of neural stimuli via a second stimulus electrode configuration according to respective values of the stimulus intensity parameter.

26. The method of claim 24, wherein the stimuli are delivered from the respective stimulus electrode configurations in an order which is the same when repeated.

27. The method of claim 26, wherein the order is configured to maximize a geometric distance between consecutively used stimulus electrode configurations.

28. The method of claim 24, wherein the stimuli are delivered from the respective stimulus electrode configurations in an order which is permuted when repeated.

29. The method of claim 28, wherein permuting the order comprises Markov sampling.

30. The method of claim 29, further comprising changing a transition probability matrix of the Markov sampling, depending on whether a preceding response intensity was zero.

31. The method of claim 24, further comprising choosing the value of the stimulus intensity parameter stochastically, between an ECAP threshold T and a discomfort threshold Max for the corresponding stimulus electrode configuration.

32. The method of claim 31, further comprising sampling the value of the stimulus intensity parameter from a uniform distribution between the ECAP threshold T and the discomfort threshold Max for the corresponding stimulus electrode configuration.

33. The method of claim 31, further comprising sampling the value of the stimulus intensity parameter from respective normal distributions each having parameters which, at a respective stimulus electrode configuration, depend dynamically on measurements from one or more preceding stimulus electrode configurations.

34. The method of claim 33, wherein a mean and a standard deviation of the respective normal distribution at each stimulus electrode configuration is initially set to a default, and wherein after stimulating at a preceding stimulus electrode configuration the mean of the distribution at a neighbouring stimulus electrode configuration is reduced if a non-zero response intensity was detected from the preceding stimulus electrode configuration or is increased if a zero response intensity was detected on the preceding stimulus electrode configuration.

35. The method of claim 33, wherein after stimulating at the preceding stimulus electrode configuration, the standard deviation of the neighbouring stimulus electrode configuration distribution is altered by an amount which is a function of the geometric distance between the stimulus electrode configurations.

36. The method of claim 31, further comprising estimating the discomfort threshold at each stimulus electrode configuration from the ECAP threshold at that stimulus electrode configuration.

37. The method of claim 36, wherein estimating the discomfort threshold at each stimulus electrode configuration comprises applying a linear model to the ECAP threshold at that stimulus electrode configuration.

38. The method of claim 31, further comprising estimating the ECAP threshold at each stimulus electrode configuration.

39. The method of claim 38, wherein estimating the ECAP threshold at a stimulus electrode configuration comprises applying a noise departure detector to detect ECAPs in captured signal windows corresponding to multiple neural stimuli of different stimulus intensity parameters delivered via the stimulus electrode configuration.

40. The method of claim 24, wherein the estimating comprises fitting an activation plot model to the plurality of (stimulus intensity parameter, response intensity) pairs for that stimulus electrode configuration.

41. The method of claim 40, wherein the activation plot model is a straight line, and one of the one or more key parameters is a slope of the fitted straight line.

42. The method of claim 40, wherein the activation plot model is a straight line, and one of the one or more key parameters is an intercept of the fitted straight line.

43. The method of claim 40, wherein the activation plot model is a logistic growth curve, and one of the one or more key parameters is a slope of the fitted logistic growth curve at its midpoint.

44-53. (canceled)
